(19) United States
(12) Patent Application Publication
(10) Pub. No.: US 2020/0260976 A1
(43) Pub. Date: Aug. 20, 2020

(54) ESTIMATING METHOD, ESTIMATING APPARATUS AND MAGNETIC RESONANCE IMAGING APPARATUS FOR ESTIMATING REGION OF NERVE ACTIVITY

(71) Applicant: CANON MEDICAL SYSTEMS CORPORATION, Otawara-shi (JP)

(72) Inventors: Naoki SASAKI, Otawara (JP); Hitoshi Yamagata, Otawara (JP)

(73) Assignee: CANON MEDICAL SYSTEMS CORPORATION, Otawara-shi (JP)

(21) Appl. No.: 16/782,133

(22) Filed: Feb. 5, 2020

(30) Foreign Application Priority Data
Feb. 19, 2019 (JP) 2019-027130
Jan. 30, 2020 (JP) 2020-013368

(51) Int. Cl. A61B 5/04; A61B 5/055; A61B 5/00

(52) U.S. Cl. CPC A61B 5/04005; A61B 5/055; A61B 5/0044; A61B 5/0042; A61B 5/4076

(57) ABSTRACT

An estimating method according to an embodiment is an estimating method for estimating a region of nerve activity, the estimation method including estimating which nerve bundle has conducted an electric current in a subject, based on information representing a three-dimensional structure of nerve bundles and information representing a distribution of a magnetic field near a surface of the subject.

### CROSS-REFERENCE TO RELATED APPLICATIONS

This application is based upon and claims the benefit of priority from Japanese Patent Application No. 2019-027130, filed on Feb. 19, 2019; and Japanese Patent Application No. 2020-013368, filed on Jan. 30, 2020, the entire contents of all of which are incorporated herein by reference.

### FIELD

Embodiments described herein relate to an estimating method, an estimating apparatus and a magnetic resonance imaging (MRI) apparatus for estimating a region of nerve activity.

### BACKGROUND

Conventionally having been known is a technology for collecting magnetoencephalography (MEG) data representing a distribution of a magnetic field near a brain surface, by measuring the magnetic field generated by an electric current resultant of nerve activity and conducted through nerve bundles in the brain, using a superconducting quantum interference device (SQUID). Research and development efforts are currently being made for a SQUID system capable of detecting a weak magnetic field without the use of a magnetically shielded room, and investigations are now being made in pursuit for a method for estimating a region of nerve activity in a brain based on MEG data.

### BRIEF DESCRIPTION OF THE DRAWINGS

FIG. 1 is a schematic illustrating an exemplary configuration of a medical information processing apparatus according to a first embodiment;

FIG. 2 is a schematic illustrating a method for calculating a magnetic field distribution, performed by a generating function according to the first embodiment;

FIG. 3 is a schematic illustrating the method for calculating the magnetic field distribution, performed by the generating function according to the first embodiment;

FIG. 4 is a flowchart illustrating the sequence of a process implemented by processing functions included in processing circuitry according to the first embodiment;

FIG. 5 is a schematic illustrating an exemplary configuration of a medical information processing apparatus according to a second embodiment;

FIG. 6 is a schematic illustrating a method for identifying an abnormal region of a brain, performed by the medical information processing apparatus according to the second embodiment;

FIG. 7 is a flowchart illustrating the sequence of a process implemented by processing functions included in processing circuitry according to the second embodiment;

FIG. 8 is a schematic illustrating an exemplary configuration of a medical information processing apparatus according to a third embodiment;

FIG. 9 is a schematic illustrating a method for estimating nerve bundles, performed by the medical information processing apparatus according to the third embodiment;

FIG. 10 is a schematic illustrating an exemplary configuration of an MRI apparatus according to a fourth embodiment; and

FIG. 11 is a schematic illustrating a method for calculating a magnetic field distribution, performed by a generating function according to a fifth embodiment.

### DETAILED DESCRIPTION

An estimating method according to embodiments is an estimating method for estimating a region of nerve activity, and includes estimating a nerve bundle having conducted an electric current, based on information representing a three-dimensional structure of nerve bundles of a subject, and on information representing a distribution of a magnetic field near a surface of the subject.

Some embodiments of an estimating method, an estimating apparatus and a magnetic resonance imaging apparatus for estimating a region of nerve activity will now be explained in detail with reference to some drawings.

### First Embodiment

In a first embodiment, an example in which the estimating method and the estimating apparatus disclosed herein are applied to a medical information processing apparatus will be explained.

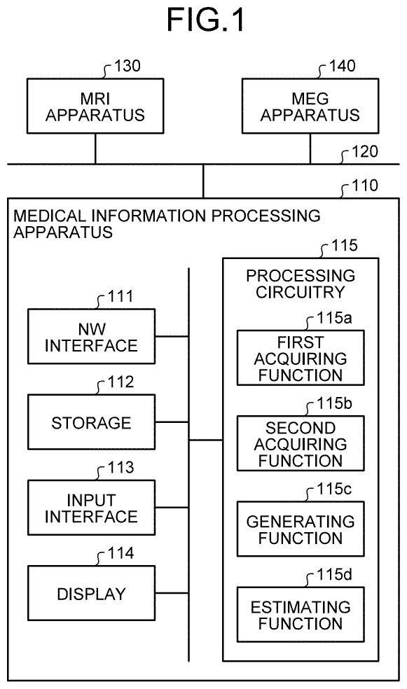

FIG. 1 is a schematic illustrating an exemplary configuration of the medical information processing apparatus according to the first embodiment.

As an example, as illustrated in FIG. 1, this medical information processing apparatus 110 according to the first embodiment is communicatively connected to apparatuses such as an MRI apparatus 130 and a MEG apparatus 140 over a network 120. For example, the medical information processing apparatus 110, the MRI apparatus 130, and the MEG apparatus 140 are installed in a hospital, and are connected to one another over a local area network (LAN) in the hospital.

The MRI apparatus 130 collects image data of a subject, by taking advantage of magnetic resonance. Specifically, the MRI apparatus 130 collects magnetic resonance data from a subject by performing various imaging sequences, based on imaging conditions specified by an operator. The MRI apparatus 130 then generates two-dimensional or three-dimensional image data by applying image processing, such as Fourier transform, to the collected magnetic resonance data.

The MEG apparatus 140 collects MEG data of the subject. Specifically, the MEG apparatus 140 collects MEG data representing a distribution of a magnetic field near the surface of the brain, by measuring the magnetic field generated by an electric current being conducted through a nerve bundle in the brain, using a SQUID.

The medical information processing apparatus 110 acquires various types of medical data from the other apparatuses connected via the network 120, and performs various types of information processing using the acquired medical data. The medical information processing apparatus 110 is implemented as, for example, a computer device such as a work station, a personal computer, and a tablet terminal.

Specifically, the medical information processing apparatus 110 includes a network (NW) interface 111, a storage 112, an input interface 113, a display 114, and processing circuitry 115.

The NW interface 111 is connected to the processing circuitry 115, and controls data communication between the medical information processing apparatus 110 and the other apparatuses over the network 120. Specifically, the NW interface 111 transmits and receives various types of data to and from the other apparatuses, under the control of the processing circuitry 115. For example, the NW interface 111 is implemented as a network card, a network adapter, or a network interface controller (NIC).

The storage 112 is connected to the processing circuitry 115, and stores therein various types of data. Specifically, the storage 112 stores therein various types of data, and reads and updates the stored data in response to a request from the processing circuitry 115. For example, the storage 112 is implemented as a random access memory (RAM), a semiconductor memory device such as a flash memory, a hard disk, or an optical disc.

The input interface 113 is connected to the processing circuitry 115, and receives input operations of various instructions and various types of information from an operator. Specifically, the input interface 113 converts an input operation received from the operator into an electric signal, and outputs the electric signal to the processing circuitry 115. The input interface 113 is implemented as, for example, a trackball, a switch button, a mouse, a keyboard, a touch pad on which an input operation is performed by touching the operation surface, a touch screen that is a display screen that is integrated with a touch pad, contactless input circuitry using an optical sensor, and voice input circuitry. In the description herein, the input interface 113 is not limited to an interface provided with a physical operation component, such as a mouse and a keyboard. The examples of the input interface 113 also include electric signal processing circuitry that receives an electric signal corresponding to an input operation from an external input device provided separately from the apparatus, and that outputs the electric signal to control circuitry.

The display 114 is connected to the processing circuitry 115, and displays various types of information and various types of data. Specifically, the display 114 converts the various types of information and the various types of data into electric signals suitable for displaying, and outputs the electric signals, in response to a request from the processing circuitry 115. The display 114 is implemented as, for example, a liquid crystal monitor, a cathode ray tube (CRT) monitor, or a touch panel.

The processing circuitry 115 controls the operation of the medical information processing apparatus 110, in response to an input operation received from the operator via the input interface 113. For example, the processing circuitry 115 is implemented as a processor.

One exemplary configuration of the medical information processing apparatus 110 according to the first embodiment has been explained above. With such a configuration, the medical information processing apparatus 110 according to the embodiment has a function for estimating a region of nerve activity in a brain, based on MEG data of the subject (subject person).

Usually, in order to identify the exact location of an abnormal nerve bundle by estimating the position of the nerve bundles in the brain and electric currents conducted through the nerve bundle from the MEG data, it is necessary to solve an inverse problem. However, because such an estimating method lacks a constraint required in solving an inverse problem, it can be said that there has never been a solution allowing an appropriate answer to be obtained.

Based on the above, the medical information processing apparatus 110 according to the embodiment is configured to enable an estimation of a region of nerve activity in a brain more accurately and efficiently.

Specifically, the medical information processing apparatus 110 according to the embodiment estimates which nerve bundles in the brain have conducted an electric current, based on information representing a three-dimensional structure of the nerve bundles in the brain, and on information representing a distribution of a magnetic field near the brain surface of the subject. In this embodiment, as the information representing a three-dimensional structure of the nerve bundles in the brain, diffusion tensor tractography (DTT) data obtained with the MRI apparatus 130 is used. Furthermore, in this embodiment, as the information representing a distribution of a magnetic field near the brain surface of the subject, MEG data obtained with the MEG apparatus 140 is used.

More specifically, in this embodiment, the processing circuitry 115 includes a first acquiring function 115a, a second acquiring function 115b, a generating function 115c, and an estimating function 115d. The first acquiring function 115a is one example of a first acquiring unit. The second acquiring function 115b is one example of a second acquiring unit. The generating function 115c is one example of a generating unit. The estimating function 115d is one example of an estimating unit.

The first acquiring function 115a acquires DTT data representing a three-dimensional structure of the nerve bundles in the brain.

Specifically, the first acquiring function 115a acquires the DTT data of a subject from the MRI apparatus 130 via the network 120. The DTT data is imaging data acquired using DTT, being acquired by keeping track of the direction of the maximum diffusion across some voxels included in imaging data acquired by diffusion tensor imaging (DTI) or Q-ball imaging (QBI), and by delineating the tracked trajectory as a nerve bundle. The DTT data is three-dimensional DTT data having a plurality of voxels including three-dimensional position information.

The second acquiring function 115b acquires MEG data representing a distribution of a magnetic field near the brain surface of a subject.

Specifically, the second acquiring function 115b acquires MEG data of the subject from the MEG apparatus 140 via the network 120. The MEG data herein is data representing a distribution of a magnetic field on a spherical surface defined by a plurality of SQUIDs placed in a manner surrounding the brain, in the MEG apparatus 140.

The generating function 115c generates a plurality of patterns of distributions of magnetic fields by calculating, for each of nerve bundles, a distribution of a magnetic field generated near the brain surface when an electric current is conducted through the nerve bundle, based on the DTT data of the subject, acquired by the first acquiring function 115a.

Specifically, the generating function 115c identifies the nerve bundles in the brain from the DTT data of the subject, and generates a plurality of patterns by calculating, for each of the identified nerve bundles, a distribution of a magnetic field generated near the brain surface when an electric current is conducted through the nerve bundle, a plurality of number of times, while changing the value of the electric current for the nerve bundle. At this time, the generating function 115c generates a pattern of a magnetic field distribution corresponding to one nerve bundle for each of the nerve bundles, under a constraint that a vector at each voxel in the DTT data is established as a current element and an electric current of the same value is conducted through the current elements included in one nerve bundle, by obtaining the magnetic field generated near the brain surface by the current elements included in the nerve bundle when an electric current is conducted through the nerve bundle, and by superimposing the magnetic fields calculated for the respective current elements.

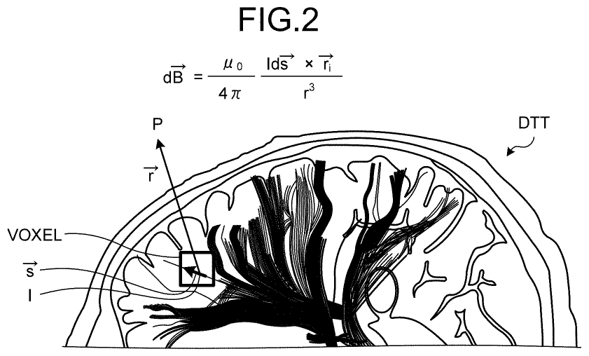

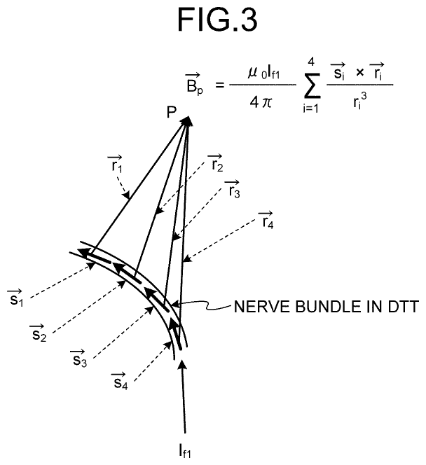

FIGS. 2 and 3 are schematics illustrating a magnetic field distribution calculated by the generating function 115c according to the first embodiment.

For example, as illustrated in FIG. 2, DTT data is mapping of vector flows achieved by connecting vectors obtained from restricted diffusion tensors, each vector of which corresponds to one voxel acquired by DTI or QBI, and in which each vector flow represents a nerve bundle. With such DTT data, establishing a vector at each voxel as a current element, a magnetic field vector generated by the current elements near the brain can be calculated based on the Biot-Savart law.

In other words, establishing a vector s at each voxel as a current element, and denoting the value of an electric current as I, it is possible to obtain a vector dB of a magnetic field generated by the current element and generated at a point P located outside of the brain at a distance of a vector r, using Equation (1) below. μ₀ herein is a magnetic permeability of the vacuum.

$$d \vec{B} = \frac{{μ}_{0}}{4 π} \frac{Id \vec{s} \times \vec{r}}{{r}^{3}} \qquad ( 1 )$$

As an example, it is assumed herein that four current elements are concatenated along one nerve bundle that is delineated in the DTT data, as illustrated in FIG. 3. Because the electric current conducted through one nerve bundle remains the same, it is possible to give a constraint that an electric current of the same value is conducted through the current elements included in one nerve bundle.

In other words, as indicated by Equation (2) below, based on the vectors s₁ to s₄ at the four respective current elements included in the nerve bundle, the vector r₁ to r₄ representing the distances of the four respective current elements to the point P, and the constant electric current I_{f1} being conducted through the nerve bundle, it is possible to calculate a vector B_{P} of the magnetic field generated at the point P, by superimposing the magnetic fields generated by these four current elements.

$${\vec{B}}_{p} = \frac{{μ}_{0} {I}_{f 1}}{4 π} {\sum }_{i = 1}^{4} \frac{{\vec{s}}_{i} \times {\vec{r}}_{i}}{{r}_{i}^{3}} \qquad ( 2 )$$

It is also possible to obtain the magnetic field generated by another nerve bundle at the point P, by doing the same calculation. It can be said that a different electric current is conducted through a current element depending on the nerve bundle to which the current element belongs.

The generating function 115c generates, for each of the nerve bundles identified in the DTT data, a plurality of patterns of distributions of magnetic fields by calculating, for each of the nerve bundles identified in the DTT data, a distribution of a magnetic field generated near the brain surface, by calculating the magnetic field distribution a plurality of number of times, following the method described above, while changing the value of the electric current for the nerve bundle. It is assumed that a pattern calculated by the generating function 115c contains not only the information representing a distribution of a magnetic field, but also information of the value of the electric current used in the calculation of the magnetic field for the nerve bundle.

The estimating function 115d estimates which nerve bundles have conducted an electric current in a brain during the period in which the MEG data is collected, based on the correlation between the patterns of the magnetic field distribution generated by the generating function 115c, and the MEG data of the subject, acquired by the second acquiring function 115b.

Specifically, the estimating function 115d identifies a pattern that matches the MEG data of the subject, acquired by the second acquiring function 115b, or a pattern nearest thereto, from the patterns of the magnetic field distribution generated by the generating function 115c. The estimating function 115d then estimates the nerve bundles having conducted an electric current during the period in which the MEG data of the subject is collected, by identifying the nerve bundles exhibiting some non-zero electric current from the identified pattern. The estimating function 115d then displays the information representing the estimated nerve bundles on the display 114, as a result of the nerve bundle estimation. For example, the estimating function 115d displays the DTT data of the subject, acquired by the first acquiring function 115a, on the display 114, and displays the estimated nerve bundles over the DTT data, in an emphasized manner.

At this time, after identifying the nerve bundles, the estimating function 115d may estimate a region (e.g., a gyrus) of the brain where the nerve bundles are concatenated. For example, the estimating function 115d estimates a brain region where the identified nerve bundles are concatenated by aligning a three-dimensional model representing a plurality of brain regions that are functional areas or anatomical areas, e.g., gyri, of a typical brain, to the DTT data by deforming the model. The estimating function 115d then displays, for example, the estimated nerve bundles and the brain region where the nerve bundles are concatenated in an emphasized manner, over the DTT image displayed on the display 114.

Alternatively, if a T1-weighted image of the subject has already been acquired with the MRI apparatus 130, for example, the estimating function 115d may display a map of cerebral functional areas, e.g., that of gyri, over the T1-weighted image, and display the nerve bundles and the brain region where the estimated nerve bundles are concatenated in an emphasized manner.

An example in which DTT data of a subject is used has been explained above, but if there is no available DTT data of a subject from which the MEG data is collected, for example, DTT data of a typical brain may be used instead. In such a case, for example, the estimating function 115d estimates a brain region where the nerve bundles are concatenated using the DTT data of a typical brain and a model of brain regions corresponding to the brain, and displays the estimated nerve bundles and the brain region where the nerve bundles are concatenated in an emphasized manner, over the DTT image. In such a case, because used is a model of brain regions corresponding to the DTT data of a typical brain, it is not necessary to perform the alignment between the DTT data and the model when the brain region is to be estimated.

The processing functions of the processing circuitry 115 have been explained above. When the processing circuitry 115 is implemented as a processor, for example, each of these processing functions is stored in the storage 112 in the form of a computer-executable program. The processing circuitry 115 implements the function corresponding to each of the computer programs by reading the computer program from the storage 112 and executing the computer program. In other words, the processing circuitry 115 having read the computer programs has the functions of the processing circuitry 115 illustrated in FIG. 1. In FIG. 1, the processing functions are explained to be implemented on one processor, but it is also possible to configure processing circuitry as a combination of a plurality of independent processors, and to implement the functions by causing the processors to execute the computer programs. The processing function of the processing circuitry 115 may be implemented in a manner distributed or integrated into one or more processing circuitries, as appropriate. Furthermore, in the example illustrated in FIG. 1, the storage 112 that is one circuitry is explained to store therein the computer programs corresponding to the processing functions, but it is also possible to deploy a plurality of storages in a distributed manner, and to cause the processing circuitry to read the corresponding computer programs from the independent storages.

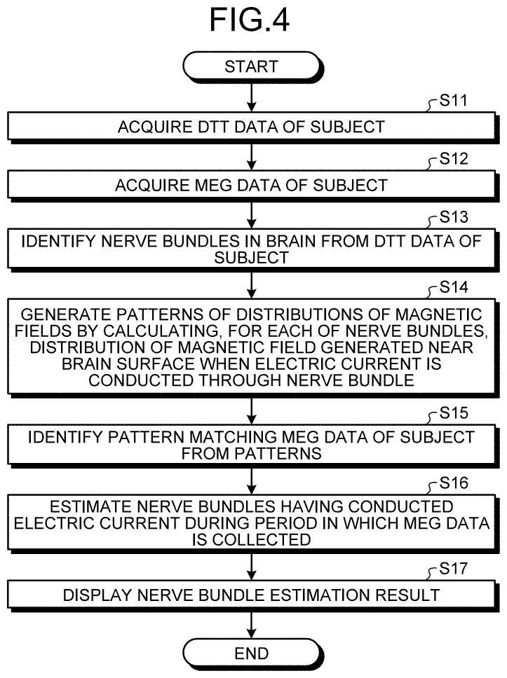

FIG. 4 is a flowchart illustrating the sequence of a process implemented by the processing functions of the processing circuitry 115 according to the first embodiment.

For example, as illustrated in FIG. 4, in this embodiment, the first acquiring function 115a acquires the DTT data of a subject from the MRI apparatus 130 (Step S11). This step is implemented by, for example, causing the processing circuitry 115 to read a predetermined computer program corresponding to the first acquiring function 115a from the storage 112, and to execute the computer program.

The second acquiring function 115b acquires MEG data of the subject from the MEG apparatus 140 (Step S12). This step is implemented by, for example, causing the processing circuitry 115 to read a predetermined computer program corresponding to the second acquiring function 115b from the storage 112, and to execute the computer program.

The generating function 115c then identifies nerve bundles in the brain from the DTT data of the subject acquired by the first acquiring function 115a (Step S13), and generates a plurality of patterns of distributions of magnetic fields by calculating, for each of nerve bundles, a distribution of the magnetic field generated near the brain surface when an electric current is conducted through the nerve bundles (Step S14). These steps are implemented by, for example, causing the processing circuitry 115 to read a predetermined computer program corresponding to the generating function 115c from the storage 112, and to execute the computer program.

The estimating function 115d then identifies a pattern matching the MEG data of the subject (or a pattern nearest thereto) from the patterns of a magnetic field distribution generated by the generating function 115c (Step S15), and estimates the nerve bundles having conducted an electric current during the period in which the MEG data is collected, based on the identified pattern (Step S16). The estimating function 115d then displays the result of the nerve bundle estimation on the display 114 (Step S17). These steps are implemented by, for example, causing the processing circuitry 115 to read a predetermined computer program corresponding to the estimating function 115d from the storage 112, and to execute the computer program.

As described above, the medical information processing apparatus 110 according to the first embodiment generates a plurality of patterns of distributions of magnetic fields by calculating, for each of nerve bundles, a distribution of a magnetic field near the brain surface generated when an electric current is conducted through the nerve bundle in the brain, based on DTT data representing a three-dimensional structure of the nerve bundles of a brain. Furthermore, the medical information processing apparatus 110 estimates which nerve bundles in the brain has conducted an electric current during the period in which MEG data representing a distribution of a magnetic field near the brain surface of the subject is collected, based on the correlation between the patterns and the MEG data.

In the manner described above, the estimation of the nerve bundles having conducted an electric current using a plurality of patterns of a magnetic field distribution and MEG data corresponds to solving an inverse problem. In this embodiment, however, because patterns of a magnetic field distribution are generated for each of the nerve bundles after the paths of the nerve bundles are identified using DTT data, it is possible to obtain an appropriate solution of the inverse problem. Therefore, according to the first embodiment, it is possible to estimate a region of nerve activity in a brain more accurately and efficiently.

### Second Embodiment

Explained above in the first embodiment is an example of estimating the nerve bundles having conducted an electric current during the period in which the MEG data is collected, based on the MEG data of the subject, but the embodiment is not limited thereto. For example, it is also possible to identify an abnormal region of the brain of a subject, by comparing the estimation results of the nerve bundles obtained with the MEG data of the subject, with the estimation results obtained with the MEG data of a healthy person. Such an example will now be explained below, as a second embodiment. In the second embodiment, points that are different from those explained in the first embodiment will be mainly explained, and detailed explanations of the points that are the same as those in the first embodiment will be omitted.

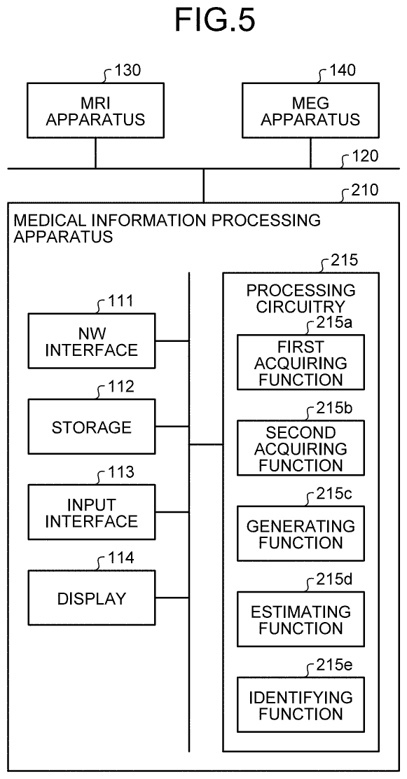

FIG. 5 is a schematic illustrating an exemplary configuration of a medical information processing apparatus according to the second embodiment.

For example, as illustrated in FIG. 5, in this medical information processing apparatus 210 according to the second embodiment, processing circuitry 215 includes a first acquiring function 215a, a second acquiring function 215b, a generating function 215c, an estimating function 215d, and an identifying function 215e. The first acquiring function 215a is one example of the first acquiring unit. The second acquiring function 215b is one example of the second acquiring unit. The generating function 215c is one example of the generating unit. The estimating function 215d is one example of the estimating unit. The identifying function 215e is one example of the identifying unit.

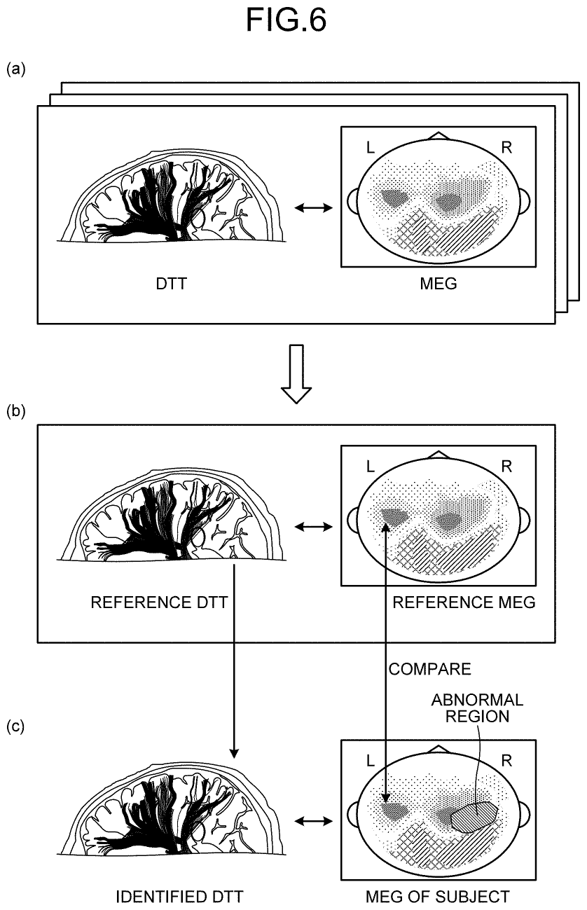

FIG. 6 is a schematic illustrating a method for identifying an abnormal region of a brain, performed by the medical information processing apparatus 210 according to the second embodiment.

In this embodiment, for example, as illustrated in portion (a) of FIG. 6, the first acquiring function 215a acquires DTT data of healthy persons belonging to each age group in the resting state, from the MRI apparatus 130. The second acquiring function 215b also acquires MEG data of the healthy persons belonging to each age group in the resting state, from the MEG apparatus 140. The second acquiring function 215b also acquires MEG data of a subject in the resting state, in the same manner as in the first embodiment.

Furthermore, for example, as illustrated in portion (b) of FIG. 6, the first acquiring function 215a generates reference DTT data for each of the age groups, by taking an average of the DTT data of the healthy persons belonging to the age group, and having been acquired from the MRI apparatus 130. Furthermore, the second acquiring function 215b generates reference MEG data for each of the age groups, by taking an average of the MEG data of the healthy persons belonging to the age group, and having been acquired from the MEG apparatus 140.

The generating function 215c then generates a plurality of patterns of distributions of magnetic fields by calculating, for each of nerve bundles, a distribution of a magnetic field a distribution of the magnetic field generated near the brain surface when an electric current is conducted through the nerve bundles in the resting state, by performing the same process as that according to the first embodiment using the reference DTT data corresponding to the age group to which the subject belongs. Furthermore, the estimating function 215d estimates the nerve bundles having conducted an electric current in the resting state, by performing the same process as that according to the first embodiment, using the patterns of a magnetic field distribution in the resting state, generated by the generating function 215c, and the reference MEG corresponding to the age group to which the subject belongs, and identifies the value of the electric current having been conducted through the nerve bundles.

The generating function 215c then generates a plurality of patterns of distributions of magnetic fields by calculating, for each of nerve bundles, a distribution of a magnetic field generated near the brain surface when an electric current is conducted through the nerve bundle in the resting state, by performing the same process as that according to the first embodiment, using the reference DTT data corresponding to the age group to which the subject belongs. At this time, the generating function 215c changes the value of the electric current with respect to an initial value set to the value of the electric current already identified as having been conducted through the nerve bundles, which have also been already identified in the resting state. Every time a pattern is generated, the generating function 215c compares the pattern generated by the estimating function 215d, with the MEG data of the subject in the resting state, acquired by the second acquiring function 215b, and identifies a pattern that matches the MEG data of the subject, or a pattern nearest thereto. In this manner, it is possible to reduce the time required in identifying a pattern of the magnetic field distribution. The estimating function 115d then estimates the nerve bundles having conducted an electric current in the resting state, by performing the same process as that according to the first embodiment, based on the identified pattern.

In this embodiment, for example, as illustrated in portion (c) of FIG. 6, the identifying function 215e then identifies the abnormal region of the subject's brain by comparing the nerve bundles resultant of the estimation based on the MEG data of the healthy persons, with the nerve bundles resultant of the estimation based on the MEG data of the subject.

Specifically, the identifying function 215e compares the nerve bundles estimated with the reference MEG data, with the nerve bundles estimated with the MEG data of the subject, and identifies the difference nerve bundles (mainly, a smaller number of nerve bundles). The identifying function 215e then identifies a brain region (e.g., a gyrus) where the identified nerve bundles are concatenated, and identifies the identified brain region as an abnormal region. The identifying function 215e then displays, for example, the identified abnormal region in an emphasized manner, over the reference DTT image displayed on the display 114.

The “resting state” herein is a state “where the subject has his/her eyes closed without thinking about anything”. A brain is known to have a default mode network (DMN), which is a connection of nerves maintaining a certain pattern of activity in the resting state. In other words, the nerve bundles keep conducting a certain level of electric current. In this embodiment, by using the reference DTT data and the reference MEG data relating to such a resting state, it is possible to achieve a more accurate nerve bundle estimation.

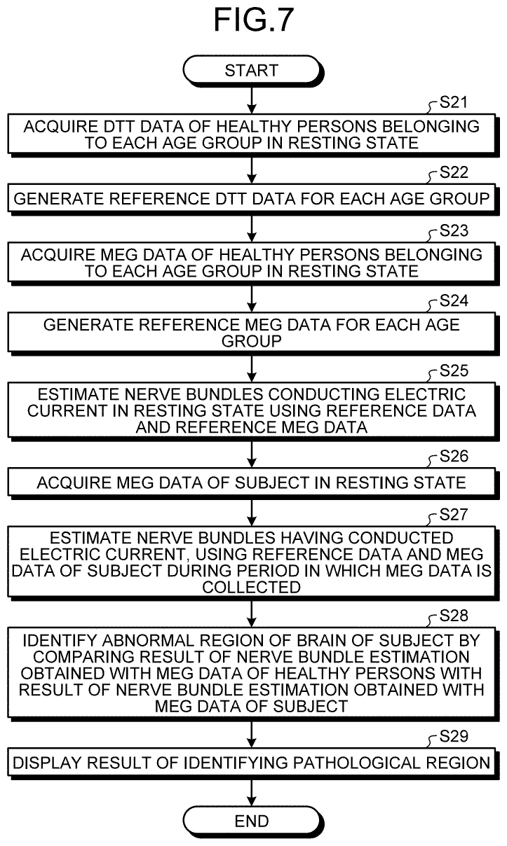

FIG. 7 is a flowchart illustrating the sequence of a process implemented by the processing functions of the processing circuitry 215 according to the second embodiment.

For example, as illustrated in FIG. 7, in this embodiment, the first acquiring function 215a acquires DTT data of healthy persons belonging to each age group in the resting state, from the MRI apparatus 130 (Step S21), and generates reference DTT data for each of such age groups (Step S22). These steps are implemented by, for example, causing the processing circuitry 215 to read a predetermined computer program corresponding to the first acquiring function 215a from the storage 112, and to execute the computer program.

The second acquiring function 215b acquires MEG data of healthy persons belonging to each of the age group in the resting state, from the MEG apparatus 140 (Step S23), and generates reference MEG data for each of the age groups (Step S24). These steps are implemented by, for example, causing the processing circuitry 215 to read a predetermined computer program corresponding to the second acquiring function 215b from the storage 112, and to execute the computer program.

The generating function 215c and the estimating function 215d then estimate the nerve bundles having conducted an electric current in the resting state, by performing the same process as that according to the first embodiment, using the reference data and the reference MEG data corresponding to the age group to which the subject belongs (Step S25). These steps are implemented by, for example, causing the processing circuitry 215 to read predetermined computer programs corresponding to the generating function 215c and the estimating function 215d from the storage 112, and to execute the computer programs.

The second acquiring function 215b then acquires the MEG data of the subject from the MEG apparatus 140 (Step S26). This step is implemented by, for example, causing the processing circuitry 215 to read a predetermined computer program corresponding to the second acquiring function 215b from the storage 112, and to execute the computer program.

The generating function 215c and the estimating function 215d then estimate the nerve bundles having conducted an electric current in the resting state, by performing the same process as that according to the first embodiment, using the reference data corresponding to the age group to which the subject belongs, and the MEG data of the subject (Step S27). These steps are implemented by, for example, causing the processing circuitry 215 to read predetermined computer programs corresponding to the generating function 215c and the estimating function 215d from the storage 112, and to execute the computer programs.

The identifying function 215e then identifies an abnormal region of the brain of the subject, by comparing the result of the nerve bundle estimation obtained with the MEG data of healthy persons, with the result of the nerve bundle estimation obtained with the MEG data of the subject (Step S28). The identifying function 215e then displays the result of identifying the abnormal region on the display 114 (Step S29). These steps are implemented by, for example, causing the processing circuitry 215 to read a predetermined computer program corresponding to the identifying function 215e from the storage 112, and to execute the computer program.

As described above, the medical information processing apparatus 210 according to the second embodiment identifies an abnormal region of the brain of a subject, by comparing the result of the nerve bundle estimation obtained with the MEG data of healthy persons, with the result of the nerve bundle estimation obtained with the MEG data of the subject. In this manner, according to the second embodiment, it is possible to identify a region of an abnormal nerve bundle and an abnormal function area, such as a gyrus, from which the nerve bundle originates, from the MEG data, accurately.

Explained in the second embodiment above is an example in which the DTT data of the resting state is used, but the embodiment is not limited thereto. Because DTT data usually does not change depending on the state of the subject, it is possible to use DTT data collected in a state that is not the resting state.

Explained in the second embodiment is an example in which used are the MEG data of healthy persons and the MEG data of a subject, all in the resting state, but the embodiment is not limited thereto. For example, the MEG data of the healthy persons and the MEG data of the subject do not necessarily need to be those in the resting state, as long as these pieces of data are those acquired in the same state. In other words, in this embodiment, it is possible to identify an abnormal region, from the MEG data acquired in various states other than the resting state.

### Third Embodiment

Explained in the first embodiment is an example for estimating the nerve bundles having conducted an electric current during the period in which MEG data of a subject is collected, based on the correlation between patterns of a magnetic field distribution calculated based on DTT data, and the MEG data, but the embodiment is not limited thereto. For example, it is also possible to estimate the nerve bundles having conducted an electric current during the period in which MEG data is collected, by performing machine learning using DTT data, MEG data, and information of the nerve bundles having conducted an electric current during the period in which the MEG data is collected, as training data. Such an example will now be explained below as a third embodiment. In the third embodiment, points that are different from those explained in the first embodiment will be mainly explained, and detailed explanations of the points that are the same as those in the first embodiment will be omitted.

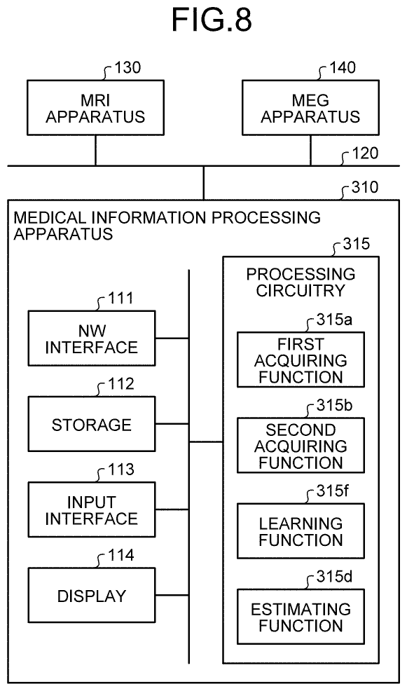

FIG. 8 is a schematic illustrating an exemplary configuration of a medical information processing apparatus according to the third embodiment.

For example, as illustrated in FIG. 8, in this medical information processing apparatus 310 according to the third embodiment, processing circuitry 315 includes a first acquiring function 315a, a second acquiring function 315b, a learning function 315f, an estimating function 315d, and an identifying function 315e. The first acquiring function 315a is one example of the first acquiring unit. The second acquiring function 315b is one example of the second acquiring unit. The learning function 315f is one example of a learning unit. The estimating function 315d is one example of the estimating unit.

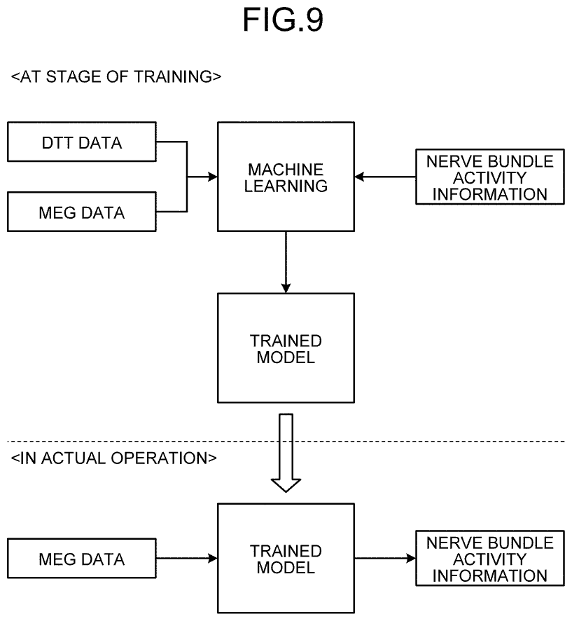

FIG. 9 is a schematic illustrating a method for estimating nerve bundles, performed by the medical information processing apparatus 310 according to the third embodiment.

FIG. 9 illustrates a process performed at the stage of training, and a process performed in the actual operation, both being performed by the medical information processing apparatus 310 according to the embodiment. In this embodiment, it is assumed herein that, as an example, the storage 112 stores therein DTT data and MEG data having been put in use in the past nerve bundle estimations, and also stores therein nerve bundle activity information representing the results of the nerve bundle estimations performed with these pieces of data.

For example, as illustrated at the top of FIG. 9, at the stage of the training, the learning function 315f acquires the DTT data and MEG data used in the past nerve bundle estimations, and the nerve bundle activity information from the storage 112. The learning function 315f then performs machine learning using the acquired DTT data, MEG data, and nerve bundle activity information, as training data.

As the machine learning performed by the learning function 315f, it is possible to use various techniques such as deep learning, logistic regression analysis, nonlinear discriminant analysis, support vector machine (SVM), random forest, and Naive Bayes.

As a result of such machine learning, the learning function 315f generates a trained model that outputs nerve bundle activity information representing the nerve bundles having conducted an electric current during the period in which a piece of MEG data of a subject is collected, based on the MEG data. The learning function 315f then stores the generated trained model in the storage 112. If the storage 112 stores therein some existing trained model having been previously created, the learning function 315f replaces the trained model being stored, with the newly created trained model.

By contrast, for example, as illustrated in the bottom of FIG. 9, in the actual operation, the estimating function 315d inputs the MEG data of the subject, acquired by the second acquiring function 315b, to the trained model generated by the learning function 315f, and estimates the nerve bundles having conducted an electric current during the period in which the MEG is collected, based on the nerve bundle activity information output as a result from the trained model. The estimating function 315d then displays the result of the nerve bundle estimation on the display 114, in the same manner as in the first embodiment.

The processes performed by the first acquiring function 315a, the second acquiring function 315b, the learning function 315f, and the estimating function 315d explained above are implemented by causing the processing circuitry 315 to read and to execute predetermined computer programs corresponding to the first acquiring function 315a, the second acquiring function 315b, the learning function 315f, and the estimating function 315d, respectively, from the storage 112, for each of such processes, for example.

As described above, the medical information processing apparatus 310 according to the third embodiment estimates the nerve bundles having conducted an electric current during the period in which MEG data is collected, by performing machine learning using DTT data, MEG data, and information of the nerve bundles having conducted an electric current while the MEG data is being collected, as the training data. In this manner, according to the third embodiment, it is possible to estimate the nerve bundles at a higher speed.

Modifications of First to Third Embodiments

It is also possible to implement the medical information processing apparatus explained in the embodiments with a part of configurations thereof modified as appropriate.

For example, when the MEG apparatus 140 supports disposition of a plurality of SQUIDs at different positions in the radial directions with respect to the brain as the center, so that the MEG apparatus 140 is capable of collecting a plurality of pieces of MEG data representing distributions of magnetic fields on a plurality of spherical surfaces sharing the same center but located at different diameters, the medical information processing apparatus may estimate the nerve bundles using the MEG data related to each of such spherical surfaces. In such a case, for example, the generating function generates patterns of a magnetic field distribution corresponding to each spherical surface, by changing the vector r of the distance between the current element and the point P, for the electric current of the same value. The estimating function then estimates the nerve bundles having conducted an electric current by comparing the patterns of the magnetic field distribution with the MEG data corresponding to each of the spherical surfaces, and identifying the pattern of the magnetic field distribution that matches completely, or that is nearest to the MEG data across all of the spherical surfaces. In this manner, the pattern of the magnetic field distribution can be identified more accurately, and it is possible to improve the accuracy of the nerve bundle measurement.

Furthermore, for example, in the embodiments described above, the generating function is explained to generate the patterns of a magnetic field distribution for all of nerve bundles identified in the DTT data, but it is also possible to configure the generating function to generate such patterns for some of the nerve bundles. In such a case, for example, the generating function identifies a region exhibiting a prominent reaction exceeding a threshold of the magnetic field in the MEG data, and generates patterns of a magnetic field distribution only for the nerve bundles that are connected to the identified region. In this manner, it is possible to reduce the processing time accrued in calculating patterns of the magnetic field distribution, so that it is possible to reduce the time required in estimating the nerve bundles.

### Fourth Embodiment

Explained in the embodiments described above is an example in which the estimating method and the estimating apparatus disclosed herein are applied to the medical information processing apparatus, but the embodiment is not limited thereto. For example, the estimating method and the estimating apparatus disclosed herein may be applied to an MRI apparatus. In the explanation below, such an example will be explained as a fourth embodiment.

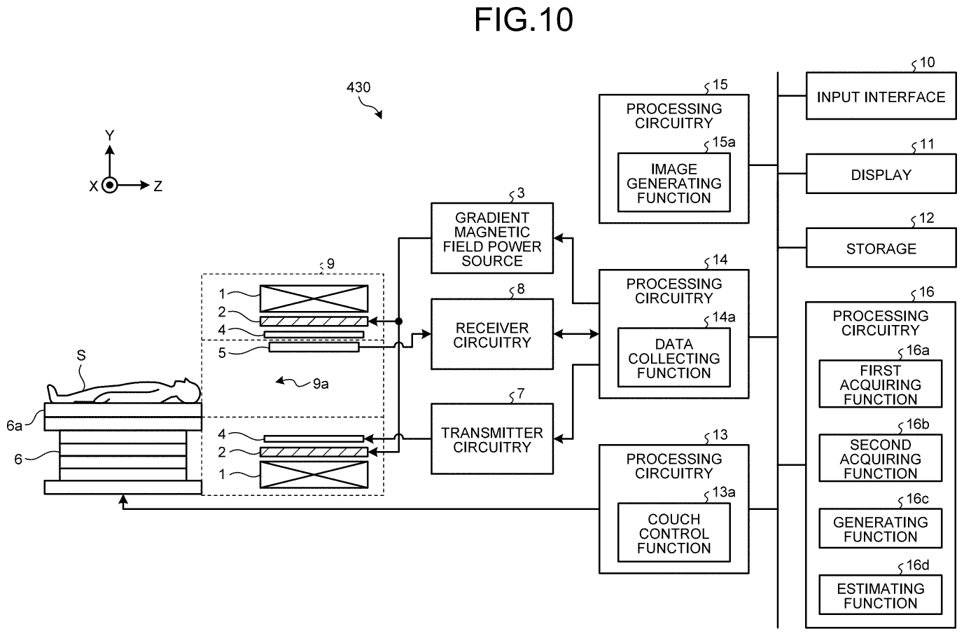

FIG. 10 is a schematic illustrating an exemplary configuration of an MRI apparatus according to the fourth embodiment.

For example, as illustrated in FIG. 10, this MRI apparatus 430 according to the embodiment include a static magnetic field magnet 1, a gradient coil 2, a gradient magnetic field power source 3, a body radio-frequency (RF) coil 4, a local RF coil 5, a couch 6, transmitter circuitry 7, receiver circuitry 8, a gantry 9, an input interface 10, a display 11, a storage 12, and processing circuitries 13 to 16.

The static magnetic field magnet 1 generates a static magnetic field in an imaging space where a subject S is laid. Specifically, the static magnetic field magnet 1 has a substantially cylindrical shape inside of which is hollow (including a shape having an elliptical cross section in a direction perpendicularly intersecting with the central axis), and generates a static magnetic field in the imaging space formed by an inner circumferential surface thereof. For example, the static magnetic field magnet 1 includes a cooling container having a substantially cylindrical shape, and a magnet such as a superconducting magnet immersed in a coolant (e.g., liquid helium) that is filled in the cooling container. The static magnetic field magnet 1 may also be configured to generate a static magnetic field using a permanent magnet, for example.

The gradient coil 2 generates a gradient field in the imaging space where the subject S is laid. Specifically, the gradient coil 2 has a substantially cylindrical shape inside of which is hollow (including a shape having an elliptical cross section in a direction perpendicularly intersecting with the central axis), and generates a gradient field in the imaging space formed by the inner circumferential surface thereof, based on the electric current supplied from the gradient magnetic field power source 3. The gradient coil 2 also includes an X coil, a Y coil, and a Z coil corresponding to an X axis, a Y axis, and a Z axis, respectively, and generates gradient fields in the imaging space, in the X axis, the Y axis, and the Z axis that perpendicularly intersect with one another, based on the electric currents supplied to the respective coils from the gradient magnetic field power source 3.

The X axis, the Y axis, and the Z axis form an apparatus coordinate system that is unique to the MRI apparatus 430. For example, the X axis is set in a manner extending along the horizontal direction, the Y axis is set in a manner extending along the vertical direction, and the Z axis is matched with the axial direction of the gradient coil 2 and is set in a manner extending along the magnetic flux of the static magnetic field generated by the static magnetic field magnet 1.

The gradient magnetic field power source 3 generates the gradient fields in the imaging space, in the directions extending along the X axis, the Y axis, and the Z axis, by supplying independent electric currents to the X coil, the Y coil, and the Z coil, respectively, provided to the gradient coil 2. In the manner described above, by causing the gradient magnetic field power source 3 to supply appropriate electric currents to the X coil, the Y coil, and the Z coil, respectively, the gradient fields can be generated in a read-out direction, a phase encoding direction, and a slice direction that perpendicularly intersect with one another.

The axis extending along the read-out direction, the axis extending along the phase encoding direction, and the axis extending along the slice direction form a logical coordinate system that defines a slice region or a volume region that is to be imaged. Specifically, by superimposing the gradient fields in the read-out direction, the phase encoding direction, and the slice direction, respectively, over the static magnetic field generated by the static magnetic field magnet 1, spatial position information is given to an MR signal emitted from the subject S. The gradient field in the read-out direction gives the position information in the read-out direction to an MR signal, by changing the frequency of the MR signal depending on the position in the read-out direction. The phase-encoding gradient field gives the position information in the phase encoding direction to the MR signal, by changing the phase of the MR signal in the phase encoding direction. The slice gradient field gives the position information in the slice direction to the MR signal. For example, when the region to be imaged is a slice region, the slice gradient field is used in deciding the direction, the thickness, and the number of the slice regions. When the region to be imaged is a volume region, the slice gradient field is used in changing the phase of the MR signal depending on the position in the slice direction.

The body RF coil 4 is an RF coil that applies an RF magnetic field to the imaging space where the subject S is laid, and that receives the MR signal emitted from the subject S due to the effect of the RF magnetic field. Specifically, the body RF coil 4 has a substantially cylindrical shape inside of which is hollow (including a shape having an elliptical cross section in a direction perpendicularly intersecting with the central axis), and applies an RF magnetic field to the imaging space formed by the inner circumferential surface thereof, based on an RF pulse signal supplied by the transmitter circuitry 7. The body RF coil 4 also receives the MR signal emitted from the subject S due to the effects of the RF magnetic field, and outputs the received MR signal to the receiver circuitry 8. For example, the body RF coil 4 is a quadrature (QD) coil.

The local RF coil 5 is an RF coil that receives the MR signal emitted from the subject S. Specifically, the local RF coil 5 is an RF coil provided for each region of the subject S, and is placed near the region to be imaged, every time the subject S is to be imaged. The local RF coil 5 receives the MR signal emitted from the subject S by being applied with the RF magnetic field, which is applied by the body RF coil 4, and outputs the received MR signal to the receiver circuitry 8. The local RF coil 5 may also have a function of a transmission coil for applying the RF magnetic field to the subject S. In such a case, the local RF coil 5 is connected to the transmitter circuitry 7, and applies an RF magnetic field to the subject S based on the RF pulse signal supplied from the transmitter circuitry 7. For example, the local RF coil 5 is a surface coil, or an array coil including a plurality of surface coils.

The couch 6 is provided with a couchtop 6a on which the subject S is laid, and moves the couchtop 6a where the subject S is laid, into the imaging space when imaging of the subject S is to be performed. For example, the couch 6 is installed in such a manner that the longitudinal direction of the couchtop 6a becomes parallel with the central axis of the static magnetic field magnet 1.

The transmitter circuitry 7 outputs an RF pulse signal corresponding to the Larmor frequency that is unique to the target nucleus placed in the static magnetic field, to the body RF coil 4. Specifically, the transmitter circuitry 7 includes a pulse generator, an RF generator, a modulator, and an amplifier. The pulse generator generates the waveform of an RF pulse signal. The RF generator generates an RF signal at a resonance frequency. The modulator generates an RF pulse signal by modulating the amplitude of the RF signal generated by the RF generator, using the waveform generated by the pulse generator. The amplifier amplifies the RF pulse signal generated by the modulator, and outputs the resultant signal to the body RF coil 4.

The receiver circuitry 8 generates MR signal data based on the MR signals received at the body RF coil 4 and the local RF coil 5, and outputs the generated MR signal data to the processing circuitry 14. Specifically, the receiver circuitry 8 has a detector, and generates MR signal data by causing the detector to subtract a resonance frequency component from the MR signals received at the body RF coil 4 and the local RF coil 5, and outputs the generated MR signal data to the processing circuitry 14.

The gantry 9 has a hollow bore 9a having a substantially cylindrical shape (including a shape having an elliptical cross section in a direction perpendicularly intersecting with the central axis), and supports the static magnetic field magnet 1, the gradient coil 2, and the body RF coil 4. Specifically, the gantry 9 supports the static magnetic field magnet 1, the gradient coil 2, and the body RF coil 4, with the gradient coil 2 positioned on the inner circumferential surface of the static magnetic field magnet 1, the body RF coil 4 positioned on the inner circumferential surface of the gradient coil 2, and the bore 9a positioned on the inner circumferential surface of the body RF coil 4. The space inside of the bore 9a of the gantry 9 forms the imaging space where the subject S is laid when imaging of the subject S is to be carried out.

Explained herein is an example of the MRI apparatus 430 in which all of the static magnetic field magnet 1, the gradient coil 2, and the body RF coil 4 have a substantially cylindrical shape, that is, what is called a tunnel-like structure, but the embodiment is not limited thereto. For example, the MRI apparatus 430 may also have what is called an open structure where a pair of static magnetic field magnets, a pair of gradient coils, and a pair of RF coils are disposed facing each other, with the imaging space where the subject S is laid interposed therebetween. In such a case, the space between the pair of static magnetic field magnets, the pair of gradient coils, and the pair of RF coils corresponds to the bore in the tunnel-like structure.

The input interface 10 receives input operations of various instructions and various types of information from an operator. Specifically, the input interface 10 is connected to the processing circuitry 16, converts the input operations received from the operator into electric signals, and outputs the signals to the processing circuitry 16. For example, the input interface 10 is implemented as, for example, a trackball, a switch button, a mouse, a keyboard, a touch pad on which an input operation is performed by touching the operation surface, a touch screen that is a display screen that is integrated with a touch pad, contactless input circuitry using an optical sensor, and voice input circuitry, for setting imaging conditions or a region of interest (ROI). In the description herein, the input interface 10 is not limited to an interface provided with a physical operation component, such as a mouse and a keyboard. The examples of the input interface 10 also include electric signal processing circuitry that receives an electric signal corresponding to an input operation from an external input device provided separately from the apparatus, and that outputs the electric signal to control circuitry.

The display 11 displays various types of information and various types of data. Specifically, the display 11 is connected to the processing circuitry 16, and converts the various types of information and the various types of data received from the processing circuitry 16 to electric signals suitable for displaying, and outputs the electric signals. The display 11 is implemented as, for example, a liquid crystal monitor, a CRT monitor, or a touch panel.

The storage 12 stores therein various types of data. Specifically, the storage 12 stores therein MR signal data and image data. For example, the storage 12 is implemented as a RAM, a semiconductor memory device such as a flash memory, a hard disk, or an optical disc.

The processing circuitry 13 has a couch control function 13a. The couch control function 13a controls the operations of the couch 6 by outputting controlling electric signals to the couch 6. For example, the couch control function 13a receives instructions for moving the couchtop 6a in the longitudinal direction, the up-and-down direction, or the left-to-right direction, from an operator via the input interface 10, and causes a mechanism for moving the couchtop 6a provided to the couch 6 to move the couchtop 6a in accordance with the received instruction.

The processing circuitry 14 has a data collecting function 14a. The data collecting function 14a collects MR signal data of the subject S by executing various pulse sequences. Specifically, the data collecting function 14a executes a pulse sequence by driving the gradient magnetic field power source 3, the transmitter circuitry 7, and the receiver circuitry 8 in accordance with the sequence execution data output from the processing circuitry 16. The sequence execution data herein is data representing a pulse sequence, and is information specifying the timing at which the gradient magnetic field power source 3 supplies an electric current to the gradient coil 2, the intensity of the electric current, the power and the timing at which the transmitter circuitry 7 supplies the RF pulse signal to the body RF coil 4, and the timing at which the receiver circuitry 8 detects the MR signal, for example. The data collecting function 14a then receives MR signal data from the receiver circuitry 8, as a result of the execution of the pulse sequence, and stores the received MR signal data in the storage 12. The set of MR signal data received by the data collecting function 14a is stored in the storage 12 as data forming a k space, by being plotted two dimensionally or three dimensionally, in accordance with the position information given by the read-out gradient field, the phase-encoding gradient field, and the slice gradient field described above.

The processing circuitry 15 has an image generating function 15a. The image generating function 15a generates an image based on the MR signal data stored in the storage 12. Specifically, the image generating function 15a generates an image by reading the MR signal data stored in the storage 12, having been stored by the data collecting function 14a, and applying a post-process, that is, a reconstructing process such as Fourier transform, to the read MR signal data. The image generating function 15a also stores the image data of the generated image in the storage 12.

The processing circuitry 16 controls the entire MRI apparatus 430 by controlling the elements of the MRI apparatus 430. Specifically, the processing circuitry 16 controls the elements of the MRI apparatus 430 by displaying a graphical user interface (GUI) for receiving input operations of various instructions and various types of information from the operator on the display 11, and controlling the elements in accordance with the input operations received via the input interface 10. For example, the processing circuitry 16 executes various pulse sequences by receiving inputs of imaging conditions from the operator via the input interface 10, generating sequence execution data based on the received imaging conditions, and transmitting the sequence execution data to the processing circuitry 14. As another example, the processing circuitry 16 reads image data from the storage 12 in response to a request of an operator, and outputs the image data on the display 11. In this embodiment, the processing circuitry 16 includes a first acquiring function 16a, a second acquiring function 16b, a generating function 16c, and an estimating function 16d, which are described later.

The processing circuitries 13 to 16 described above are implemented as a processor, for example. In such a case, the processing function provided to each of the processing circuitries is stored in the storage 12 in the form of a computer-executable program, for example. Each of the processing circuitries implements the function of the corresponding computer program by reading the computer program from the storage 12, and executing the computer program. Each of the processing circuitries may include a plurality of processors, and the processing functions may be implemented by causing each of the processors to execute a computer program. Furthermore, the processing functions provided to the processing circuitries may be implemented in a manner distributed to or integrated into one or more processing circuitries. Furthermore, explained herein is an example in which the storage 12 that is one circuitry stores therein the computer programs corresponding to the respective processing functions, but it is also possible to deploy a plurality of storages in a distributed manner, and to cause the processing circuitry to read a computer program from corresponding one of the storages.

An exemplary configuration of the MRI apparatus 430 according to the fourth embodiment has been explained above. With such a structure, the MRI apparatus 430 according to the embodiment has a function for estimating a region of nerve activity in a brain based on the MEG data of a subject (subject person).

The MRI apparatus 430 according to the embodiment is configured in such a manner that it is possible to estimate a region of nerve activity in a brain more accurately and efficiently, in the same manner as with the medical information processing apparatus 110 explained in the first embodiment.

Specifically, the MRI apparatus 430 according to the embodiment estimates the nerve bundles having conducted an electric current in a brain, based on information representing a three-dimensional structure of the nerve bundles in the brain and information representing a magnetic field distribution near the brain surface of the subject. In this embodiment, as the information representing a three-dimensional structure of the nerve bundles in the brain, DTT data collected by the MRI apparatus 430 is used. Furthermore, in this embodiment, as the information representing the magnetic field distribution near the brain surface of the subject, MEG data acquired by the MEG apparatus is used.

More specifically, in this embodiment, the processing circuitry 16 includes the first acquiring function 16a, the second acquiring function 16b, the generating function 16c, and the estimating function 16d. The first acquiring function 16a is one example of the first acquiring unit. The second acquiring function 16b is one example of the second acquiring unit. The generating function 16c is one example of the generating unit. The estimating function 16d is one example of the estimating unit.

The first acquiring function 16a has the same function as that of the first acquiring function 115a explained in the first embodiment, except that the first acquiring function 16a according to the embodiment acquires the DTT data from the storage 12, while the first acquiring function 115a according to the first embodiment acquires the DTT data via a network.

The second acquiring function 16b has the same function as that of the second acquiring function 115b explained in the first embodiment. Specifically, the second acquiring function 16b acquires MEG data from a MEG apparatus connected to the MRI apparatus 430 via a network (not illustrated in FIG. 10).

The generating function 16c has the same function as that of the generating function 115c explained in the first embodiment. The estimating function 16d has the same function as that of the estimating function 115d explained in the first embodiment.

In this embodiment, the storage 12, the input interface 10, and the display 11 correspond to the storage 112, the input interface 113, and the display 114, respectively, that are explained in the first embodiment.

The processes performed by the first acquiring function 16a, the second acquiring function 16b, the generating function 16c, and the estimating function 16d explained above are implemented by causing the processing circuitry 16 to read predetermined computer programs corresponding to the first acquiring function 16a, the second acquiring function 16b, the generating function 16c, and the estimating function 16d, respectively, from the storage 12, and to execute the computer programs, for example.

With the structure described above, with the MRI apparatus 430 according to the fourth embodiment, it is possible to estimate a region of nerve activity in a brain more accurately and efficiently, in the same manner as the medical information processing apparatus 110 explained in the first embodiment.

Explained in the fourth embodiment described above is an example in which the processing circuitry 16 in the MRI apparatus 430 has the same function as that of the processing circuitry 115 explained in the first embodiment, but the embodiment is not limited thereto. For example, the processing circuitry 16 in the MRI apparatus 430 may have the same function as that of the processing circuitry 215 explained in the second embodiment, the same function as that of the processing circuitry 315 explained in the third embodiment, or the function explained in the modification of the first to the third embodiments.

### Fifth Embodiment

Explained in the embodiments described above is an example in which a region of nerve activity in a brain is estimated, but the embodiment is not limited thereto. For example, in the first embodiment, it is possible to estimate the region where a nerve-stimulating electric current has propagated in a myocardium, by using DTT data of the myocardium instead of DTT data of a brain, and using magnetocardiography (MCG) data instead of MEG data. In the explanation below, such an example will be explained as a fifth embodiment. In this embodiment, myofibers in a myocardium correspond to nerve bundles.

Specifically, in this embodiment, the first acquiring function 115a acquires DTT data representing a three-dimensional structure of myofibers in a myocardium.

At this time, because the shape of the heart changes periodically as the heart pulsates, in order to identify the three-dimensional structures of the myofibers more accurately, it is preferable to use the DTT data collected during the period in which the amount of change in the shape of the heart is small, in the cardiac cycle. For example, DTT data collected in a diastolic period, in which the movement of the left ventricle is the smallest, is used.

The second acquiring function 115b acquires magnetocardiography (MCG) data representing a distribution of a magnetic field near the surface of the myocardium of a subject.

The MCG data is collected, for example, by causing a SQUID to measure the magnetic field generated by the electric currents being conducted through Purkinje fibers, which are myofibers in a cardiac conduction system that is present in a myocardium, when stimuli are applied to the myocardium of the subject using a pacemaker, for example. The MCG data used herein is data collected correspondingly to the DTT data in the diastolic period.

Furthermore, the generating function 115c generates a plurality of patterns of distributions of magnetic fields by calculating, for each of nerve bundles, a distribution of a magnetic field generated near the surface of the myocardium when an electric current has been conducted through the myofibers, based on the DTT data acquired by the first acquiring function 115a, following the same method as that used in the first embodiment.

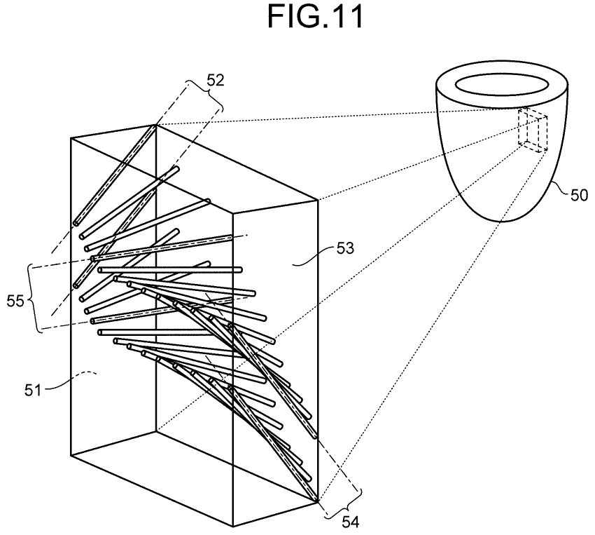

FIG. 11 is a schematic illustrating a method for calculating the magnetic field distribution, performed by the generating function 115c according to the fifth embodiment.

For example, as illustrated in FIG. 11, it is known that the myocardium in the left ventricle 50 of a heart has three layers: an inner layer of right-handed helical myofibers 52 on the side of the endocardium 51; an outer layer of right-handed helical myofibers 54 on the side of the epicardium 53, and a middle layer of circular myofibers 55 disposed between the right-handed helical myofibers 52 and the right-handed helical myofibers 54. In the DTT data of the myocardium, a plurality of myofibers in each of these layers are delineated as vector flows that are connections of vectors each corresponding to one voxel.

In this embodiment, establishing a vector at each voxel of the DTT data as a current element, the generating function 115c calculates vectors of the magnetic field generated by the current element near the myocardium based on the Biot-Savart law, in the same manner as in the first embodiment. The generating function 115c also generates, for each of the myofibers identified in the DTT data, a plurality of patterns of distributions of magnetic fields by calculating, for each of nerve bundles, a distribution of a magnetic field generated near the surface of the myocardium by calculating the magnetic field distribution a plurality of number of times, while changing the value of the electric current for the myofiber.

The estimating function 115d then estimates which myofibers in a myocardium have conducted an electric current during the period in which the MCG data is collected, based on the correlation between the patterns of the magnetic field distribution generated by the generating function 115c, and the MCG data of the subject, acquired by the second acquiring function 115b, following the same method as that used in the first embodiment.

In this manner, according to the embodiment, it is possible to estimate the regions of nerve activity in a myocardium more accurately and efficiently.

Furthermore, for example, by using DTT data of myocardium instead of DTT data of a brain, and using MCG data instead of MEG data in the second embodiment, it is possible to identify an abnormal region of the myocardium of a subject.

Furthermore, for example, by using DTT data of a myocardium instead of the DTT data of a brain, and using MCG data instead of MEG data in the third embodiment, it is also possible to estimate the myofibers having conducted an electric current during the period in which the MCG data is collected, using machine learning.

Furthermore, for example, by using DTT data of a myocardium instead of DTT data of a brain, and MCG data instead of MEG data in the fourth embodiment, it is possible to estimate the region where a nerve-stimulating electric current has propagated in a myocardium, using an MRI apparatus.

Explained in the embodiments described above is an example in which the first acquiring unit, the second acquiring unit, the generating unit, the estimating unit, the identifying unit, and the learning unit explained herein are implemented as the first acquiring function, the second acquiring function, the generating function, the estimating function, the identifying function, and the learning function of the processing circuitry, but the embodiment is not limited thereto. For example, the first acquiring unit, the second acquiring unit, the generating unit, the estimating unit, the identifying unit, and the learning unit explained herein may be implemented using only hardware, or a combination of hardware and software, in addition to the implementation as the first acquiring function, the second acquiring function, the generating function, the estimating function, the identifying function, and the learning function described in the embodiments.

The term “processor” used in the above explanation means, for example, a central processing unit (CPU), a graphics processing unit (GPU), or an application-specific integrated circuit (ASIC), a programmable logic device (e.g., a simple programmable logic device (SPLD), a complex programmable logic device (CPLD), and a field-programmable gate array (FPGA)). The processor implements a function by reading a computer program stored in the storage, and executing the computer program. Instead of storing the computer program in the storage, the computer program may be incorporated into the processor circuitry directly. In such a case, the processor implements a function by reading a computer program incorporated in the circuitry, and executing the computer program. Furthermore, the processor according to the embodiments may also be a processor configured as a combination of a plurality of independent circuitries, without limitation to the configuration as single circuitry, and to implement the function.

A computer program executed by the processor is provided in a manner incorporated in a read-only memory (ROM) or a storage, for example. This computer program may also be provided in a manner recorded in a computer-readable storage medium such as a compact disc read-only memory (CD-ROM), a flexible disk (FD), a compact disc recordable (CD-R), or a digital versatile disc (DVD), as a file in a format installable to or executable on these apparatuses.

Furthermore, this computer program may be stored in a computer that is connected to a network such as the Internet, and may be provided by making it available for download or distributed over the network. For example, this computer program has a modular structure including the functional units described above. As actual hardware, by causing a CPU to read the computer program from a storage medium such as a ROM, and executing the computer program, the modules are loaded onto the main memory, and generated on the main memory.

According to at least one of the embodiments explained above, it is possible to estimate nerve activity in a subject more accurately and efficiently.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An estimating method for estimating a region of nerve activity, the estimation method comprising:
estimating which nerve bundle has conducted an electric current in a subject, based on information representing a three-dimensional structure of nerve bundles and information representing a distribution of a magnetic field near a surface of the subject.

2. The estimating method according to claim 1, wherein
the information representing the three-dimensional structure is diffusion tensor tractography data,
the information representing the distribution of a magnetic field is magnetographic data, and
the estimating of the nerve bundle includes:
generating a plurality of patterns of distributions of magnetic fields by calculating, for each of nerve bundles, a distribution of a magnetic field generated near the surface when an electric current is conducted through the nerve bundle, based on the diffusion tensor tractography data; and
estimating which of the nerve bundles have conducted an electric current during a period in which the magnetographic data is collected, based on a correlation between the patterns and the magnetographic data.

3. The estimating method according to claim 1, wherein the nerve bundles are nerve bundles in a brain of the subject.

4. The estimating method according to claim 1, wherein the nerve bundles are myofibers in a myocardium of the subject.

5. The estimating method according to claim 2, wherein the generating of the plurality of patterns of distributions of magnetic fields includes:
identifying the nerve bundles from the diffusion tensor tractography data, and
generating the patterns by calculating, for each of the identified nerve bundles, a distribution of a magnetic field generated near the surface when an electric current is conducted through the nerve bundle, a plurality of number of times, while changing a value of the electric current for the nerve bundle.

6. The estimating method according to claim 5, wherein the generating a plurality of patterns of distributions of magnetic fields includes:
generating a pattern of a distribution of a magnetic field corresponding to one nerve bundle for each of the nerve bundles, under a constraint that a vector at each voxel in the diffusion tensor tractography data is established as a current element and an electric current at a same value is conducted through current elements included in one nerve bundle, by obtaining a magnetic field generated near the surface by the current elements included in the nerve bundle when an electric current is conducted through the nerve bundle, and by superimposing magnetic fields calculated for the respective current elements.

7. The estimating method according to claim 2, further comprising:
identifying an abnormal region in the subject by comparing a result of a nerve bundle estimation performed with magnetographic data of the subject, with a result of a nerve bundle estimation performed with magnetographic data of a healthy person.

8. The estimating method according to claim 7, wherein magnetographic data of the subject in a resting state is used as the magnetographic data.

9. An estimating apparatus for estimating a region of nerve activity, the estimating apparatus comprising processing circuitry configured to:
estimate which nerve bundle has conducted an electric current in a subject, based on information representing a three-dimensional structure of nerve bundles and information representing a distribution of a magnetic field near a surface of the subject.

10. The estimating apparatus according to claim 9, wherein
the information representing the three-dimensional structure is diffusion tensor tractography data,
the information representing the distribution of a magnetic field is magnetographic data, and
the processing circuitry is configured to:
generate a plurality of patterns of distributions of magnetic fields by calculating, for each of nerve bundles, a distribution of a magnetic field generated near the surface when an electric current is conducted through the nerve bundle, based on the diffusion tensor tractography data; and
estimate which of the nerve bundles have conducted an electric current during a period in which the magnetographic data is collected, based on a correlation between the patterns and the magnetographic data.

11. The estimating apparatus according to claim 9, wherein the nerve bundles are nerve bundles in a brain of the subject.

12. The estimating apparatus according to claim 9, wherein the nerve bundles are myofibers in a myocardium of the subject.

13. A magnetic resonance imaging apparatus comprising processing circuitry configured to:
collect magnetic resonance data from a subject; and
estimate which nerve bundle has conducted an electric current in the subject, based on information representing a three-dimensional structure of nerve bundles, the information being generated based on the magnetic resonance data, and information representing a distribution of a magnetic field near a surface of the subject.

14. The magnetic resonance imaging apparatus according to claim 13, wherein
the information representing the three-dimensional structure is diffusion tensor tractography data,
the information representing the distribution of a magnetic field is magnetographic data, and
the processing circuitry is also configured to:
generate a plurality of patterns of distributions of magnetic fields by calculating, for each of nerve bundles, a distribution of a magnetic field generated near the surface when an electric current is conducted through the nerve bundle, based on the diffusion tensor tractography data; and
estimate which of the nerve bundles have conducted an electric current during a period in which the magnetographic data is collected, based on a correlation between the patterns and the magnetographic data.

15. The magnetic resonance imaging apparatus according to claim 13, wherein the nerve bundles are nerve bundles in a brain of the subject.

16. The magnetic resonance imaging apparatus according to claim 13, wherein the nerve bundles are myofibers in a myocardium of the subject.
